(19) United States
(12) Patent Application Publication
(10) Pub. No.: US 2009/0297668 A1
(43) Pub. Date: Dec. 3, 2009

(54) System and Methods for Automatic Preparation of Substitute Food Items

(76) Inventor: Homaro R. Cantu, Chicago, IL (US)

Correspondence Address: Valauskas & Pine LLC, 150 South Wacker Drive, Suite 620, Chicago, IL, 60606, US

(21) Appl. No.: 12/224,159

(22) PCT Filed: Jan. 16, 2007
(86) PCT No.: PCT/US2007/001014

§ 371 Date: Nov. 17, 2008

Related U.S. Application Data
(60) Provisional application No. 60/759,677, filed on Jan. 17, 2006.

(51) Int. Cl. A23P 1/00; G01N 33/02; A23L 1/48; G06F 7/00

(52) U.S. Cl. 426/88; 426/87; 426/231; 426/383; 700/213

(57) ABSTRACT

A food emulator for the production of an edible article that can substitute for a food item and that includes a carrier element formed of an edible material and an information element disposed on one or more surface of the carrier element. The one or more information element includes information related to the food item. The information may be one or more of a flavoring agent, a coloring agent or a texturing agent, for example. Different types and combinations of information may be provided by way of the information element and/or the carrier element to substitute for food items.

This application claims the benefit of U.S. Provisional Application No. 60/759,677, filed Jan. 17, 2006.

### FIELD OF THE INVENTION

The present invention generally relates to a system and methods for the preparation of an edible item that can convey certain information regarding, but not all of the aspects and characteristics of a different food item. Advantageously, the edible item of the present invention is a simplified version of, and can act as a substitute for the other food item. The substitute food item of the present invention may be used, for example, to convey information, such as the flavor, texture, size, shape, some or all of the dimensions or appearance of, or other aspects or characteristics regarding a food item that given the context in which the substitute food item is to be used, cannot be prepared, stored, or served, or which consumers are unable to ingest because of health, dietary, or other recommendations or restrictions. More specifically, the present invention is directed to a system and methods by which the substitute food item may be prepared generally automatically upon conveyance of preparation instructions—either by generally direct contact with or from a remote location—to a substitute food item preparation apparatus.

### BACKGROUND OF THE INVENTION

Food items are typically prepared through the application of one or more food preparation techniques or cooking processes to one or more food components including slicing, peeling, grating, mashing, aging, brewing, distilling, mixing, carbonating, fermentation, cooling, or freezing or warming, steaming, boiling, roasting, sauteing, frying, grilling, barbecuing, or broiling. While a consumer may wish to ingest the food item, the consumer may be unable to do so because, for example, the consumer does not have the necessary food components or the time, means, or skill to apply the necessary techniques to prepare the food item.

Because of the organic origin of some or all of the its components, the freshness of a food item typically degrades in quality with time. With time, the dimensions or appearance of the food item typically also changes—such as because the food item is drying out or the components of the item are consolidating or settling. Once prepared, a food item typically must be stored in some way to permit it to be consumed as intended. To slow the degradation process, and in order to maintain freshness, food items are typically placed in closed or sealed containers or within packaging with one or more barriers that are intended, for example, to prevent the loss of moisture from the food or the degradation of fat in the food by UV light or the entry of bacteria, and/or stored or maintained to lessen the exposure of the food items to uncontrolled environmental conditions. Such closed or sealed containers or packaging, or environmental control systems are expensive and add costs to the use of the food item. Conditions at times do not permit the use of protective storage. Without a source of power, conventional refrigerators have little, if any utility. As a result, consumers are prevented from ingesting the food item in a variety of conditions and contexts.

Certain food items may be unknown or unfamiliar to consumers. Consumers may wish to try such one of such food items but are reluctant or unable to do so because, for example, of the difficulty in preparing or cost of the food item, or the general unavailability of the food item to the consumer. Promoters may wish to introduce of a new food item to consumers but, given the size of the target group, cannot afford to actually prepare and serve a sample of the actual food item to each member of the group. Promoters may wish to encourage consumers to try a new food item through the use of what appears to be a well known food item but may have no easy and inexpensive means by which to do so. For example, a promoter may wish to introduce a new food item—a new apple flavored liqueur—to consumers and may wish to do so through the use of an item that has the familiar size, shape, general dimensions, or appearance of an apple but with the flavor of the new liqueur, but has no easy means to do so.

Other food items may be familiar, possibly all too familiar to certain consumers. Due to dietary or health restrictions, the consumers are required to avoid these food items. Given the lack of availability of a non-restricted version of the food item, consumers satisfy their craving for a given restricted food item by ingesting it, contrary to the advice they receive or even their better judgment. Religious or other restrictions may prevent a consumer from ingesting a food item.

Consumer may wish to have certain food items that can be prepared and served with one or more food items that may not be available. Other food items may have a characteristic such as a taste, flavor, or texture that a user may wish to mask or heighten through the use of one or more food items that may not be generally available.

It is clear that there is a demand for a version of food items that conveys certain information about the actual food item but does not have the storage characteristics, risks, or restricted components associated with the food item. It is clear that there is also a demand for a system and methods by which a version of food items can be prepared generally automatically upon conveyance of preparation instructions—either by generally direct contact with or from a remote location—to a substitute food item preparation apparatus. The present invention satisfies these various demands.

### SUMMARY OF THE INVENTION

The system and methods of the present invention are directed to the preparation of a substitute food item. The term “substitute food item” (and variations of this term) for purposes of this application means an edible item by which certain information about a food item can be conveyed to consumers without necessarily requiring the preparation or service of the food item. The term “food item” identifies that which is being simulated through the use of the substitute food item and is any item that may be consumed or received as food, drink, or a nutritional or health supplement. The term “information” or “sensory information” for purposes of this application means actual sensory or organoleptic aspects, features, or characteristics of the food item including the color, texture, flavor, smell, dimensions, or appearance of the food item or the arrangement of the food components that form the food item. Relative to the actual food item, the substitute food item is of a simplified construction that permits a user to convey information about a food item to consumers in a variety of contexts in which the food item may not necessarily be able to be prepared, stored, or served. Certain embodiments of the substitute food item may be prepared from simplified components that may not have the storage and maintenance requirements associated with the food item. The substitute food item may also take into consideration and alter, modify, mask, or balance certain aspects or features of the actual food item that are not necessarily desirable.

One embodiment of the substitute food item according to the present invention includes a carrier element and an information element. The carrier element is of a composition that it is edible. The carrier element may be of a simplified construction and/or form—such as in the shape of a sheet. The carrier element may be of a construction, form, and/or composition such that its shape or dimensions change with further exposure or processing. Further processing includes exposure to conditions that permit the carrier element to dry. The carrier element may be made of one or more edible material, including but not limited to carbohydrates, fats, proteins, and gels.

Certain embodiments of the carrier element do not necessarily convey information about a food item but may simply function as a substrate or layer on which the information element is placed and are intended, at the very least, not to mask the information that is to be conveyed through the information element. Other embodiments of the carrier element may impart or come to impart information about a food item, such as the one or more flavors, textures, colors, shape, or dimensions of the food item. These information conveying embodiments of the carrier element are intended to interact or combine with the information conveyed by the information element to produce a desired combined flavor, shape, or appearance when the consumer views, receives, and/or ingests the substitute food item. Various shapes and forms are contemplated, for example, a planar shape, circular, spherical, conic, or even shapes and forms of common items, for example, a paperclip.

The information element of the substitute food item according to the present invention conveys “sensory information” or “organoleptic information” about a food item such as the color, texture, flavor, dimensions, or appearance of the food item or the arrangement of the food components that form the food item. The information element may also take into consideration a feature or aspect of a food item that may not necessarily be considered desirable by consumers and include an aspect, feature, or quality that masks, alters, or balances the given feature or aspect. The information element is formed from one or more information materials—any one or all assisting in the conveyance of information to the user or consumer through the information element—having a composition and consistency such that it preferably can be applied to and generally retained on or below an outer surface of the carrier element and thereby forming the information element. Alternatively, the information element may be formed separately and applied generally as a unit and fixed to the carrier element. Fixation may be facilitated through a variety of means including the use of edible substance that acts as an edible adhesive.

Depending on the intended use of the substitute food item, the information element may be formed also through the use of a liquid having a composition and consistency that permits the information material to be sprayed, rolled, or swabbed, rubbed, or abraded onto or within the pores opening onto the outer surface of the carrier element. The carrier element may also be conveyed or positioned such that the carrier element may be dipped in and retain the desired information material in liquid form to produce the information element. Alternatively, the liquid information material intended to form the information element may be poured over the carrier element. Additionally, the information element may, at least in certain embodiments may be formed by drawing information material onto the carrier element through the use of a suction pulled through portions of or the entire carrier element. The carrier element may have a construction and/or composition that facilitates the formation of the information element. For example, certain embodiments of the carrier element may have a generally open porous structure to facilitate the reception of the information material within the pores and below the surface or surfaces of the carrier element such that one or more processing steps—such as the use of a suction pulled through the pores of the carrier element—may be used to draw the information material onto the carrier element. Drying also may be used to allow a liquid form of the information material to assume a relatively stable position as an information element relative to the carrier element. Among the other forms of the information material are those that are in powder or particulate form, thereby permitting the information element to be formed, for example, by dropping, blowing, or sifting the information material onto or within the carrier element.

The simplified construction of the substitute food item allows certain embodiments to be folded in various configurations as well as smoothed over, or ironed, such that it has the ability to be used multiple times. For example, a user or consumer can utilize the substitute food item as a sheet of paper but also consume It when desired.

One or both of the carrier and information elements may include a hydrocolloid emulsion including a water and/or oil soluble flavoring. The hydrocolloid may be in a gel form which is separate from the carrier element and information element.

The substitute food item may provide information regarding the food item through the use of, for example, certain, but not all components of the food item, derivatives of components of the food item, or artificial substitutes for components of the food item. Those aspects or characteristics that the substitute food item does not include may be those which are selected for exclusion such as those which a consumer cannot ingest for health, dietary, religious, or other reasons or those which cause the food item to spoil or lose characteristics inherent to the given type of food or can only be produced through cooking or processing that, for whatever reason, cannot be provided in the context in which the substitute food item is to be used. Embodiments of the substitute food item may include some but not all the dimensions or proportions of the food item, thereby permitting the substitute food item to be used in contexts in which the food item could not be used due to its size or dimensions. The substitute food item may also be of largely the same dimensions as the food item for which it substitutes in order to convey, for example, the message of the similarity of the substitute food item to the food item.

The substitute food item may communicate information beyond the food item itself. Certain embodiments of the substitute food item change color dependent upon temperature, for example, the color red for a temperature range of 100° F. to 105° F. A change in color based on a specific temperature range allows for various uses of the substitute food item, beyond delivery of information, for example, a user or consumer can apply the food item to determine body temperature, communicating a fever or requirement for medication and/or pharmaceuticals.

Additional embodiments of the present invention include more than one carrier element and/or more than one information element. Each such element may include one or more aspects or characteristics of a certain food item or a group of such food items, thereby providing a user with flexibility as to which one or more aspects, features, or characteristics of the food item or group of food items the user wishes to convey to consumers through the use of the substitute food item. The one or more carrier elements may be fixed to one or more information elements by transmogrification. Thus) more than one element can be stretched (amoebaesque), distended, collapsed, smashed or crushed into and/or with one another.

Other embodiments of the present invention include an edible infrared material positioned on the carrier element and/or information element. The infrared material allows the substitute food item (or user or consumer) to be tracked, or positionally located, using an infrared tracking device, for example a camera.

Embodiments of the present invention permit one or a planned range of substitute food items to be prepared generally automatically upon conveyance of preparation instructions—either by generally direct contact with or from a remote location—to a substitute food item preparation apparatus termed, for purposes of this application, a “food emulator”.

Other embodiments of the present invention further include an attaching means such that the substitute food item can be easily secured and removed from other surfaces. The attaching means, for example a Velcro®-like structure, hooks, adhesive, snap, or zipper to name a few, may be edible and/or degradable.

One advantage of the substitute food item of the present invention is that it permits, for example, the flavor or texture of or other information concerning a food item to be conveyed to consumers without the actual preparation, storage, or service of the food item.

Another advantage of the substitute food item of the present invention is that it permits consumers to ingest a version of a food item that the consumers are unable to consume because of health, dietary, religious, or other restrictions and thereby satisfy an interest (or cravings) that the consumers have for the food item.

A further advantage of the substitute food item of the present invention is that it permits consumers to acquaint themselves with food items which they may have never ingested before because of the general lack of availability or time or cost associated with the preparation or service of the food item.

An additional advantage of the substitute food item of the present invention is that it permits a user to introduce or promote the use of or advertise the purchase of a food item, in environmental or business conditions that do not necessarily permit the preparation or service of the actual food item. This condition may include magazines, consumer and/or trade shows, street level marketing, direct mail, vending machines, and so on.

One advantage of the preparation system and methods of the present invention is that one or a planned range of substitute food items may be prepared generally automatically upon conveyance of preparation instructions—either by generally direct contact with or from a remote location—to the food emulator of the present application.

A primary object of the present invention is to provide an edible item that can substitute for an actual food item.

An additional object of the present invention is to provide methods of preparing an edible item that can substitute for an actual food item to a certain intended degree.

An added object of the present invention is to provide a system or methods of preparing an edible item that can substitute for one or a planned range of substitute food items generally automatically upon conveyance of preparation instructions—either by generally direct contact with or from a remote location—to a food emulator.

### BRIEF DESCRIPTION OF THE DRAWINGS

The preferred embodiments of the invention will be described in conjunction with the appended drawings provided to illustrate and not to the limit the invention, where like designations denoted like elements, and in which:

FIG. 1 is a perspective view of one embodiment of a carrier element of a substitute food item according to the present invention;

FIG. 2 is a side view of an embodiment of a substitute food item according to the present invention;

FIG. 2A1 is a side view of an embodiment of a carrier element including a use component included therein;

FIG. 2A2 is a side view of the embodiment of the carrier element shown in FIG. 2A1 after being subjected to processing;

FIG. 2B1 is a perspective view of another embodiment of a substitute food item according to the present invention;

FIG. 2B2 is a perspective view of the embodiment of the substitute food item shown in FIG. 2B1 and subjected to further processing;

FIG. 3 is a perspective view of another embodiment of a carrier element showing positive surface elements and positive surface area according to the present invention;

FIG. 4 is a perspective view of an additional embodiment of a carrier element showing negative surface elements and a negative surface area according to the present invention;

FIG. 5 is a side view of another embodiment of a substitute food item including two information elements and a carrier element according to the present invention;

FIG. 6 is a side view of an additional embodiment of a substitute food item including a plurality of information elements and a carrier element according to the present invention;

FIG. 7 is a side view of an added embodiment of a substitute food item including a carrier element and two information elements with a binding element therebetween according to the present invention;

FIG. 8 is a side view of an a further embodiment of a substitute food item including two information elements positioned below and two information elements positioned above a carrier element according to the present invention;

FIG. 9 is a side view of an added embodiment of a substitute food item including a hydrocolloid emulsion surface over a carrier element on which is positioned an information element according to the present invention;

FIG. 10A is a side view of an embodiment of a system by which certain information material may be applied to a carrier element to form a substitute food item according to the present invention;

FIG. 10B is a side view of an embodiment of a system by which certain additional information material may be applied to a carrier element to form a substitute food item according to the present invention;

FIG. 10C is a side view of an embodiment of a system by which certain added information material may be applied to a carrier element to form a substitute food item according to the present invention;

FIG. 11A is an overhead view of an embodiment of the carrier after the application of the information material 51A1 according to the system shown in FIG. 10A;

FIG. 11B is an overhead view of an embodiment of the carrier after the application of the added information material 51B1 according to the system shown in FIG. 10B;

FIG. 11C is an overhead view of an embodiment of the carrier after the application of the additional information material 51C1 according to the system shown in FIG. 10C;

FIG. 12 is a front view of an embodiment of a substitute food item according to the present invention;

FIG. 13 is a back view of the embodiment of the substitute food item according to the present invention shown in FIG. 12;

FIG. 14 is a front view of another embodiment of a substitute food item according to the present invention;

FIG. 15 is a side view of an embodiment of a food emulator for production of a substitute food item according to the present invention;

FIG. 16 is a side view of another embodiment of a food emulator for production of a substitute food item according to the present invention;

FIG. 17 is a side view of an additional embodiment of a food emulator for production of a substitute food item according to the present invention;

FIG. 18 is a side view of an added embodiment of a food emulator for production of a substitute food item according to the present invention; and,

FIG. 19 is a side view of a further embodiment of a food emulator for production of a substitute food item according to the present invention.

### DETAILED DESCRIPTION OF PREFERRED EMBODIMENTS

A substitute food item according to the present invention is shown and identified in the accompanying drawings as 21. The substitute food item 21 generally includes a carrier element 31 and an information element 61.

Carrier element 31 is of a structure and has a composition such that the carrier element 31 can accept at least the information element 61. Carrier element may be of a generally uniform composition of a certain type of edible substance or a mixture or conglomeration of edible substances. Substances from which the carrier element 31 may be made include a variety of starches including soybean starch, modified food starch, corn starch, rice starch, alginate starch, cassava, tapioca, or potato starch. So that these substances can be formed into and function as the intended carrier element 31, other substances may be added, such as water, food gums, salt, rapeseed oil, microcrystalline cellulose, various forms of hydrocolloid emulsions, maltodextrin, dextrose; or other sugars, for example. In certain embodiments of the carrier element composed largely of one or more starch compositions it is preferred that sufficient water or vegetable oil is included to avoid cracking or failure of the carrier element. A carrier element 31, having relatively higher moisture content permits the shape of the carrier element to be formed and/or adjusted in size and shape. Advantageously, by varying the type of starch, the texture of the carrier element 31 may be controlled. For example, tapioca starch provides a fine-grained structure to a carrier element 31 formed therefrom.

Embodiments of the carrier element 31 may include no information about a food item and function largely as a substrate for the information element 61. Alternatively, the carrier element 31 may include information about the food item. For example, the carrier element 31 may include a natural or artificial flavoring, or a texture, or a color largely identical or reminiscent of the food item. At the very least, whatever information is carried by the carrier element 31 does not mask, and preferably supports the information carried by the information element 61.

While the carrier element 31 may be formed from a largely homogenous mixture of processed food substances, the carrier element may include also portions of food substances that did not result from extensive processing. For example, the carrier element 31 may include small portions of a food obtained by chopping, slicing, shredding, or mashing a food. These small portions may be included in a mixture with the processed food—such as a starch-based mixture—to permit the carrier element to be formed easily.

In further embodiments, the either or both the carrier element 31 and information element 61 may include vitamins, sweeteners, hygroscopic agents, binders, medicaments, bulking agents, fillers and other food adjuncts or additives.

The use of a starch based composition for the carrier element 31 advantageously allows the carrier element 31 to be easily formed in a variety of shapes and sizes. While the carrier element 31, as well as the information element 61 are shown in the accompanying drawings as being in a similar sheet-like size and shape, the carrier element and information element may be formed in different sizes and shapes and may have sizes and shapes such that, for example, a three dimensional replica of the food item is formed. One example of such a substitute food item that is intended to largely replicate the food item is a substitute food item apple that has the three dimensions of an apple but may have few, if any of the organic portions of a food item apple, thereby permitting the substitute food item apple to be used in a wide range of applications including those in which long term storage is necessary.

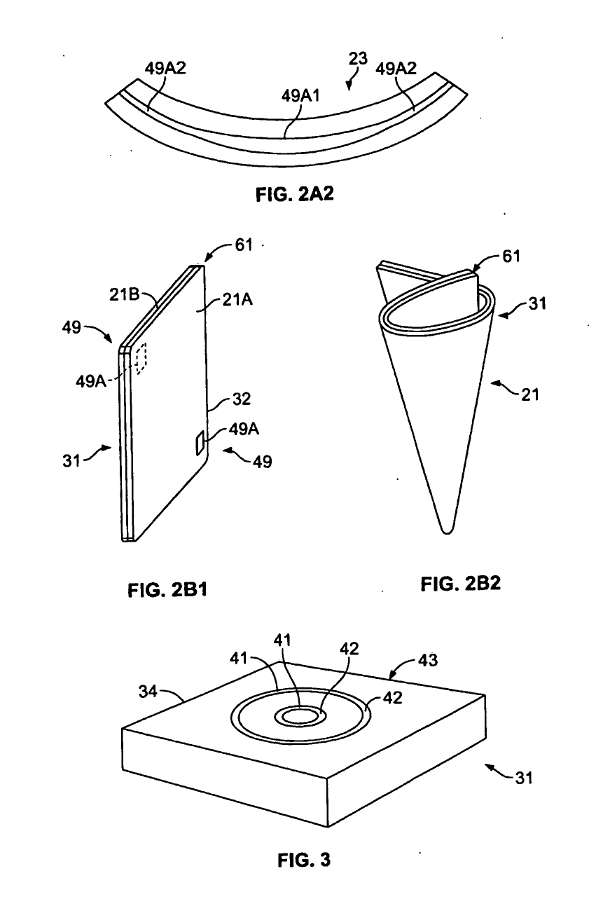

To facilitate the use and/or formation thereof, including the shaping of the carrier element 31 and thereby the entire substitute food item 21, the carrier element 31 and/or the information element 61 may include one or more use components 49. Embodiments of the use components 49 may provide support to the carrier element during and/or after formation thereof or permit the shaping of the carrier element 31, information 51, and/or some or all of the substitute food item 21. Use components 49 include structural elements 49A which may be in the form of a web, lattice, sheet, basket, network, and the like that facilitate the use of, for example, the carrier element 31, and thereby the substitute food item 21. One embodiment of a use component 49 is shown in FIG. 2 and includes structural elements 49A formed as a web of compressed food starch for supporting the relatively less dense carrier element 31 that permits the carrier element 31 to be placed in a certain configuration during processing and retained therein afterwards. The carrier element 31 may thus be held in position and supported during the application of the information material 51 such as by a silk screening or sifting process (not shown).

Another embodiment of a use component 49 includes structural elements 49A formed from one or more food substances and positioned within or on the carrier element 31 and that facilitates, for example, the shaping of the carrier element 31 or the information element 61 or the entire substitute food item 21. An example is illustrated in FIG. 2A1 and FIG. 2A2.

The use component 49 illustrated in FIG. 2A1 is of a size and shape and is positioned within the carrier element 31 such that when the formation of the substitute food item 21 is largely completed—for example, by the placement of information material 51 on or within the carrier element 31—the substitute food item 21 may be subjected to further processing—such as, given the use of a use component 49 that shrinks upon the loss of moisture, exposing the substitute food item 21 to drying conditions. Such processing permits the illustrated substitute food item 21 illustrated in FIG. 2A1 to form a shaped food item 23 as shown in FIG. 2A2 because of the relative proportions of the structural elements 49A: the thinner peripheral areas 49A2 dry more quickly than the thicker central area 49A1 and therefore shrinks quicker causing differential pulling of the substitute food item 21 into a general convex shape.

FIG. 2B1 and FIG. 2B2 illustrate another embodiment of a use component 49 having a size, shape, and composition and positioned on opposing surfaces 21A and 21B of the substitute food item 21 so that, upon further processing, the substitute food item 21 can generally hold its new processed shape 23. More specifically, the use component 49 includes structural elements 49A positioned on the opposing surfaces 21A, 21B so that after the generally planar shaped substitute food item 21 shown in FIG. 2B1 is spiraled along a common “y” axis the substitute food item 21 forms and can be held in the conic shape 23 shown in FIG. 2B2. To permit such shape retention, the use component 49 may be formed from a generally adhesive edible substance. In the conic shape 23 shown in FIG. 2B2, the substitute food item 21 may receive a food therein. The substitute food item 21 may provide information so that the combination of the substitute food item and the food carried therein mimics a well known dish. For example, the substitute food item 21 may have the flavor of a fish, such as cod, and when fried potatoes or “chips” are placed in the conic shape 23, a serving of “fish and chips” is available to consumers even when no fish is actually in the serving. The substitute food item 21 may include flavoring or have information material 51 placed thereon so that the substitute food item 21 has, for example, the taste of catsup, or vinegar and salt, or mayonnaise, or other condiment, thereby complementing the french-fries or chips food placed therein.

The substitute food item 21 may include one or more use components 49 to facilitate its further shaping or use including additional structural elements 49A that permit additional processing so that the substitute food item 21 may be shaped and used as a more closed container or apparatus—including a bottle—for the receipt of one or other food items such that the substitute food item 21 as well as the food item may be consumed.

To facilitate the conveyance of the carrier element 31 through a non-manual processing step in which the information material 51 from which the information element 61 is formed is placed on the carrier element 31, for example, through a printing or spraying process, a carrier element 31 having a generally planar shape may be particularly advantageous. The spraying process may include a fluid with solids based on edible materials including carbohydrates, food gums, aldetols, sugars, starches, proteins, fats, emulsifiers, and ethanol. These materials may be used to adjust the viscosity of the fluids used in printing or spraying of the information materials 51. Ethanol may be included to speed up drying.

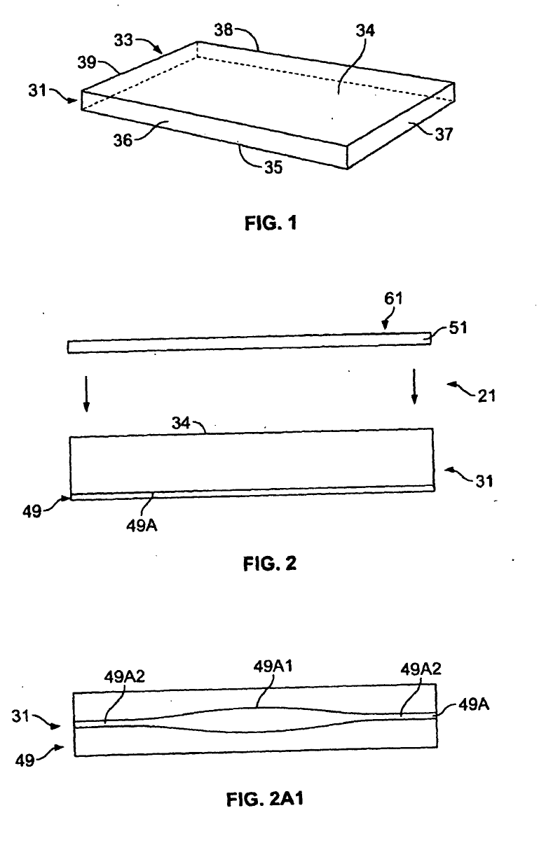

An example of a planar shaped carrier element 31 is illustrated in the accompanying drawings. The illustrated embodiment of the carrier element 31 includes an outer surface 33 including opposing major surfaces 34 and 35 and edge surfaces 36, 37, 38, and 39 (see FIG. 1).

The information element 61 conveys “information” and in particular may convey sensory or organoleptic information about a separate food item such as the color, texture, flavor, shape, dimensions, or appearance of the food item or the arrangement of the food components that form the food item. For example, if the substitute food item 21 is to convey information about an actual food item, a lemon cake, for example, the information element may have a lemon yellow color and natural or artificial flavoring that conveys a lemon flavor. The information element 61 may be supported on a carrier element 31 largely without flavor but with possibly additional information such as a texture that approximates the texture of the lemon cake. If the separate food item is a lemon cake having coconut frosting, the substitute food item 21 may include an information element 61 having, in addition to a yellow color and a lemon flavor, a natural or artificial coconut flavoring. The information element 61 may also have a texture that in part has a “mouth feel” of shredded coconut. Alternatively, the carrier element 31 may have a texture that conveys the “mouth feel” of shredded coconut. Each of these flavors and the texture may be formed within a single information element 61 that, for example, is applied to the outer surface 33 of the carrier element 31. As discussed below, embodiments of the substitute food item 21 may include a plurality of information elements or carrier elements, each of which may include certain but not all of the information of a food item.

When certain information about the food item may not necessarily be desirable, but is included within the substitute food item to in order to more closely simulate the food item, the information element 61 (or carrier element 31) may include aspects, features, or qualities to alter, modify, or partially mask the non-desirable feature. For example, if a food item has a “gamey” taste, while the substitute food item 21 may have an artificial flavor that mimics the “gamey” taste it may include another flavor that is intended to at least partially mask the flavor of the food item.

Rather than including all of the information that the user wishes to convey about a food item in a single information element 61, one or more characteristics, features, or aspects may be included in separate information elements and the information elements joined together and positioned on the carrier element 31 to form the substitute food item 21. Such embodiments permit one or more substitute food items to be prepared of one or more of food items within a larger group of food items to be formed as needed and quickly.

For example, and with reference to FIG. 6, if a user wishes to be able to prepare substitute food items of one or more food items that are related in that each has one flavor in common—such as different types of lemon cake—, the lemon flavor may be included in one information element 61A1, while the other aspects by which the other members of the group of lemon cakes are distinguishable may be included in separate information elements. The texture information of the other food items within this group of food items may be included in another and separate information element 61B1, while some other characteristic of the separate food item (such as color) may be included in additional information element 61C1, etc. The information elements 61A1, 61B1, 61C1, etc. may joined or adhered together to form a single information element 61. This separation of information of the food item into separate information elements allows different food items to be “constructed” by selection and joining of separate information elements together.

As a further example of the use of the present invention to form a substitute for a food item, a version of lemon cake with butter crème frosting can be formed, for example, and again with reference to FIG. 6, from an information element 61A1 that is yellow in color, an information element 61B1 that includes a natural or artificial lemon flavoring, and, an information element 61C1 that provides possibly the flavor and/or smooth “mouth feel” of butter crème icing, all of which are joined together and positioned on the carrier element 31. Another food item within the group of lemon cakes—for example, a lemon cake with coconut butter crème frosting—may be formed, for example, and with reference to FIG. 8, from an information element 61A1 that is yellow in color, an information element 61B1 that includes a natural or artificial lemon flavoring, an information element 61C1 that has the smooth “mouth feel” of butter crème frosting, and an information element 61D1 that has a natural or artificial coconut flavor. This embodiment may include an additional information element 61E1 (not shown in FIG. 8) that provides a texture with a mouth feel similar to that of shredded coconut. Alternatively, the texture of shredded coconut may be simulated in the carrier element of this embodiment. All of these information elements 61A1 through 61D1 may be joined together in one group information element 61 and placed on the carrier element 31.

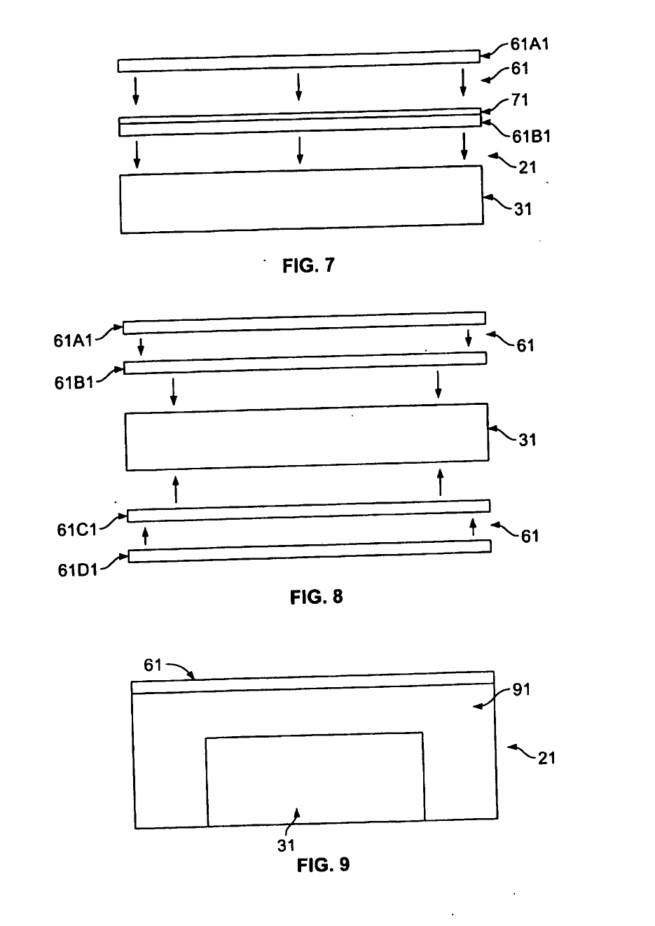

In many applications, where the information element 61 is positioned on the carrier element 31 is not of particular importance. However, in certain applications, the information elements 61 whether in single form or in multiple form, such as information elements 61A1, 61B1, etc. may be positioned on the carrier element to further simulate the actual food item. For example, if the food item that is to be simulated by the substitute food item 21 is the alcoholic drink “margarita”, one information element—such as 61A1—may include at least a grainy texture that simulates the salt that is often placed on the rim of the glass in which the drink is served while the flavor of the drink may be included in one or more additional information elements 61B1, 61C1, etc. FIG. 7 shows an embodiment of a substitute food item that may be used to simulate a margarita in which information element 61A1 has a grainy texture, and possibly a salty flavor, while the remaining flavor of the drink is included in information element 61B1. The two information elements 61A1 and 61B1 may be joined together with an edible adhesive layer 71 and the information element 61 is placed on the carrier element 31.

FIG. 9 shows an embodiment of a food substitute 21 wherein the carrier element 31 is coated or attached to a gel layer 91. The gel layer 91 may be a hydrocolloid gel, including food flavorings, sweeteners or other materials 51. An information element 61 is attached to the gel layer 91. The gel layer 91 may function to promote adherence or binding of the information element 61 to the carrier element 31. The combined carrier element 31 and gel layer 91 may be processed to promote adherence, for example, by steaming or other treatments.

FIG. 5 shows an embodiment of a substitute food item 21 in which one information element 61A1 is carried on an upper surface 34 of the carrier 31 while another information element 61B1 is carried on the lower surface 35 of the carrier 31. Such positioning may assist in the simulation of a food in which separation of strong flavors or textures in a food item is known. For example, and with reference to FIG. 5, information element 61A1 may provide a lemon color and flavor, the carrier element may have a texture simulating that of a cake, while information element 61B1 may have a smooth texture and crime flavor, thereby creating a substitute for a crème filled lemon tart.

The information element 61 is of a composition and consistency such that it preferably can be applied to and generally retained on an outer surface of the carrier element 31. The information element 61 may be formed separately and as a unit applied to and fixed to the carrier element. Fixation may be facilitated through a variety of means including the use of edible substance that acts as an edible adhesive. Depending on the intended use of the substitute food item, the information element may be formed also through the use of a liquid having a composition and consistency that permits it to be sprayed, rolled, or swabbed onto the outer surface of the carrier element.

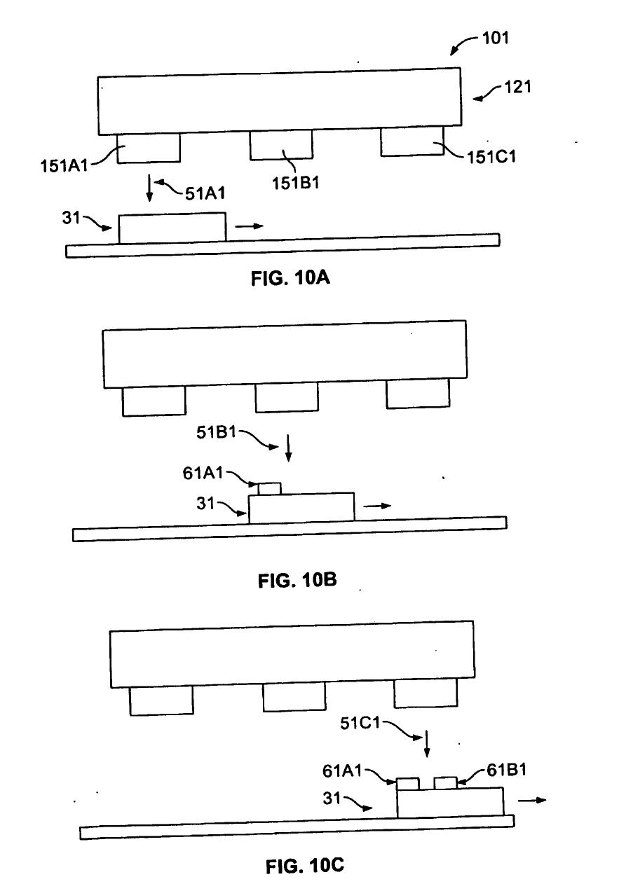

FIG. 10A, FIG. 10B, and FIG. 10C show one method 101 by which information material 51 may be applied to form an information element 61 on a carrier element 31. Specifically, the method permits different information material 51 to be applied to the carrier in a sequence. FIG. 10A shows information material 51A1 being discharged from one discharge unit 151A1 included within the application system 121 onto carrier 31. The system 121 may be a printing system, a silk screen system, or a system by which the information material 51A1 is applied in a dry form, for example, by sifting onto the carrier element 31. FIG. 10B shows information material 51B1 being discharged from a second discharge unit 151B1 (see FIG. 10A) included within the application system 121 onto the carrier 31 which has been moved from a position below the discharge unit 151A1 to the new second position under the discharge unit 151B1 either manually or automatically such as through a conveyor system. FIG. 10C shows information material 51C1 being discharged from a third discharge unit 151C1 (see FIG. 10A) included within the application system 121 onto the carrier which has already received material 51A1 from discharge unit 151A1 and material 51B1 from discharge unit 151B1 and has been moved into the new third position manually or by a conveyor-like process.

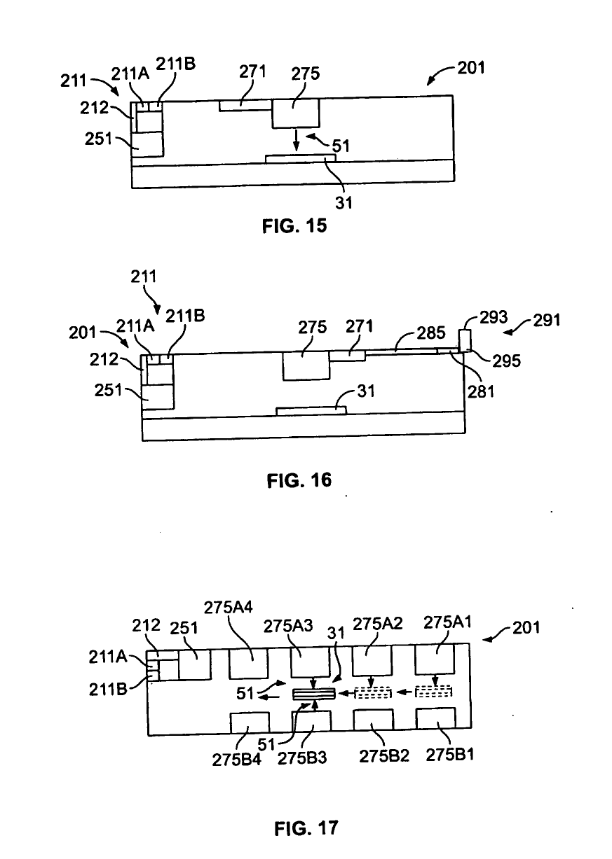

Embodiments of the preparation system and methods of the present invention permit one or a planned range of substitute food items 21 to be prepared generally automatically upon conveyance of preparation instructions—either by generally direct contact with or from a remote location—to a food emulator 201. Embodiments of the automatic preparation system and methods that utilizes a food emulator 201 by which a substitute food item may be prepared automatically upon receipt of preparation instructions 251 are shown in FIG. 15 through 19. The illustrated embodiments of the food emulator 201 permit the application of one or more information material 51 for form an information element 61 on a carrier element 31 according to the preparation instructions received by the food emulator 201 through a communication element 211.

FIG. 15 illustrates one embodiment of a food emulator 201 which includes a communication element 211 that permits a user or another or others to convey preparation instructions to the food emulator 201 and, in certain embodiments, for the food emulator to convey information back to the user or users. The communication element 211 may be in the form of a control panel 212 which permits a user to identify preferences for the type of substitute food item 21 which the user wishes the food emulator 201 to produce. In such an embodiment, the emulator 201 would include a memory component 251 that houses some or all of the instructions to control the operation of the emulator 201 so that the substitute food item 21 which the user identified through the use of the control panel 212 may be prepared. The control panel 212 may also permit the user to actually program the food emulator 201 so that substitute food item 21 is produced according to the specific preparation instructions provided by the user. Rather than, or in addition to the control panel 212, the communication element 211 may include also a receiver 211A and transmitter 211B by which the food emulator may receive preparation instructions from a user or another or others that are in a location so that physical contact with a control panel is not possible or is not preferred. Embodiments of such a food emulator 201 may permit a user or users to convey preparation instructions through a variety of communication devices including through hard wire and/or wireless devices, such as WiFi, cell phones, PDAs, and cable and Bluetooth systems.

Embodiments of the food emulator 201, such as the one shown in FIG. 15 include at least one information material storage unit 271 from which information material 51 may be drawn for placement to form an information element 61 on or within the carrier element 31. The system by which the material is applied to the carrier element 31 within the emulator 201 may be as shown in FIG. 10A through 10C and be a printing system, a silk screen system, or a system by which the information material 51 is applied in a dry form—for example by sifting onto the carrier element 31 or pulling the material 51 onto or into the carrier element 31 through the use of a suction pulled through a side (e.g., side 35) of the carrier element 31 opposite to the side (e.g., side 34) along which the material 51 may be discharged—or in wet form. FIG. 15 illustrates a discharge unit 275 which includes information material 51 or includes or can communicate with the information material storage unit 271.

Another embodiment of the food emulator 201 is shown in FIG. 16 and includes an intake component 291 that permits information material 51 or one or more components of or an entire food item (for example, for which the food emulator 201 will produce a substitute food item 21) to be received and held therein for possible use. The FIG. 16 embodiment can be used in applications in which a user wishes to convey to another or others that will be sampling or consuming the substitute food item 21 of the similarity or likeness of the substitute food item 21 to the food item placed in the intake component 291. The user can place the material 51 or food item through an intake area 293. The intake component 291 can be of a composition and structure such that food item can be held in the intake component 291 and no aspect, characteristic, or component of the food item can be conveyed to and/or used within the food emulator 201. Such an embodiment advantageously reinforces the message that the substitute food item 21 is similar to the food item placed in the intake component 291 without any component of the food item actually being used in the production of the substitute food item. Certain embodiments of the intake component 291 may include an intake control 295 which permits the user to allow some characteristic or all of the food item to enter the food emulator 201. For example, a user may wish to use the fragrance of a food item (such as crushed citrus fruit) or the steam produced by a heated food item (such as a broiled steak) in the production of the substitute food item 21 (such as, with respect to the fragrant food item, a substitute food item 21 intended to mimic a citrus-flavored cake, ice cream, or sherbet, or in the case of the steaming food item, a substitute food item 21 that mimics a broiled steak but is formed without the use of meat protein). In embodiments of the food emulator 201 that can be used with an intake component 291 having an intake control 295, the food emulator 201 may include a processor 281 by which one or more characteristics of or the entire food item may be processed to allow only the desired characteristic or characteristics of the food item to be selected and conveyed through a passage 285 in the food emulator 201 for use to form the substitute food item 21. The processor 281 may as simple as a filter but may also include an identifier 281A by which the presence and identity of the food item may be identified to the control panel 212 and the identity used during the control of the process by which the substitute food item 21 is made. In the FIG. 16 embodiment, the processor 281 is shown as communicating with the information storage unit 271, thereby allowing the component of the food item to be held until needed for discharge through the discharge unit 275.

Certain embodiments of the food emulator 201 include at least one discharge unit 275A for discharge of information material 51 on one side of the carrier element 31—such as on side 34—and another discharge unit 275B for discharge of information material 51 on the opposing side of the carrier element 31—such as on opposing side 35. Discharge units 275—such as the described discharge units 275A and 275B—permit the discharge of and transfer of information material 51 to or within the surface or surfaces of a carrier element through a variety of processes and may include structures such as sprayers, rollers, drums, and sifters. FIG. 17 illustrates an embodiment of the food emulator 201 that includes a plurality of opposing discharge units 275A1, 275A2, 275A3, 275A4 and 275B1, 275B2, 275B3, 275B4 such that one or a plurality of information materials 51 may be discharged onto and produce single or multiple layers of information elements 61 on each side 34, 35 of carrier element 31. FIG. 17 shows the carrier element in part in broken lines to show the sequential processing of it by the discharging of one or more information materials 51 on it.

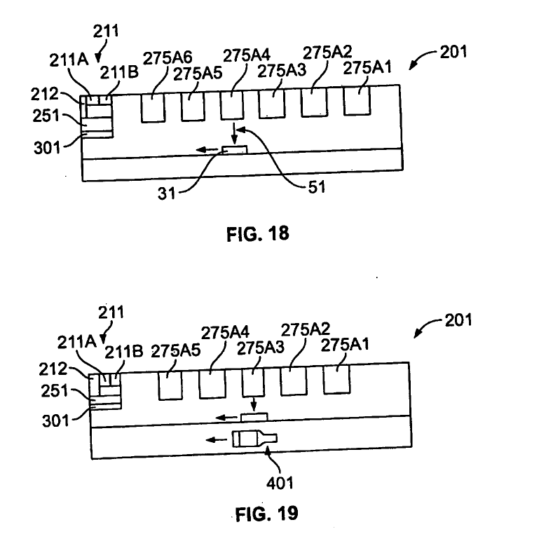

FIG. 18 shows an embodiment of a food emulator 201 that may be used to vend substitute food items 21 such as in a grocery store to provide shoppers some idea of what a food item may taste like, or in those contexts in which conventional vending machines are used. The embodiment of the food emulator 201 shown in FIG. 18 may include a carrier element that may or may not include information material 51 on or within it. Based upon instructions received from the control panel 212—such as when the shopper pushes a button to vend a substitute food item 21—the embodiment of the food emulator 201 shown in FIG. 18 may process a carrier element 31 by applying information material or materials 51 to a surface thereof by discharge from one or more discharge units 275A1, 275A2, 275A3, 275A4, 275A5, and/or 275A6. The information material 51 that is applied may be that which is particularly fragrant and easily volatilizes, and so can only be applied on order, or that which oxidizes or spoils easily, and so must be held in discharge units—that may be refrigerated—until a substitute food item 21 is ordered. The food emulator 201 may include sets of discharge units 275 positioned and structured—such as described above—to permit the application of information material 51 to additional sides of the carrier element 31.

The food emulator 201 may include means—termed for purposes of this application a recorder 301—by which the consideration received or to be received for the vending of the substitute food item 21 may be accepted or recorded. The recorder 301 may accept currency or a coupon or deduct credit from a card and thereby permit the emulator 201 to vend a substitute food item 21 for one or more types of consideration. The choice of which substitute food item 21 is vended may be selected by the consumer by use of the control panel 212 that embodiments of the emulator 201 may include. The recorder 301 may record consideration that may be as simple as number of substitute food items 21 vended by the food emulator 201 to consumers. As a result, the consideration received is a measure of the goodwill generated through the use of emulator 201.

FIG. 19 shows another embodiment of the food emulator 201 with which a substitute food item 21 may be produced. The illustrated embodiment of the food emulator 201 can be used in conjunction with a food item 401 vended also through or provided in association with the food emulator 201. The food item 401 is represented in FIG. 18 as a bottle but may be any food item that may be vended and whose flavor, taste, or other characteristics may be complemented or altered through the use of the substitute food item 21. For example, the food emulator 201 may permit a consumer to select—such as through use of a control panel 212—a substitute food item 21 that can be used with the food item 401. The substitute food item 21 may be flavored so that it changes the flavor of a food item—such as a drink. Advantageously, through the use of such a food emulator 201 a substitute food item 21—such as one that is cherry flavored and appears to look like a cherry or one that is lemon flavored and appears to look like a lemon—may be produced as needed and vended at the same time with, for example, a cola or lemon bottled beverage 401. The production of the substitute food item 21 may comprise the addition of information material 51—such as a cherry flavoring or a lemon flavoring—to a carrier element 31 and the substitute food item 21 vended at the time the food item 401 is vended.

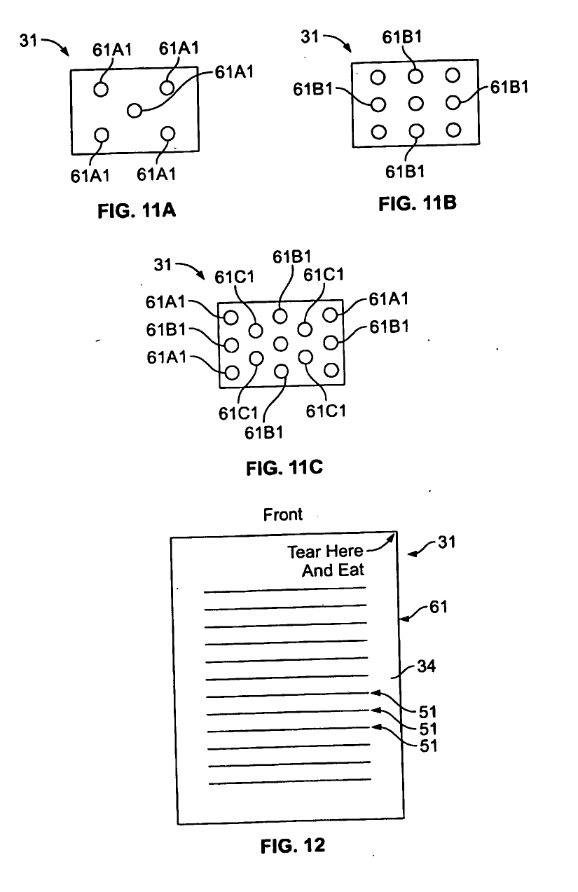

FIG. 11A, FIG. 11B, and FIG. 11C show the results of the use of the method 101 shown in FIG. 10A-10C. While the information material 51A1, 51B1, 51C1 may be applied in each step to form individual layers 61A1, 61B1, and 61C1 on the carrier element 31, the information material 51 may be applied to form less continuous sections including as the individual sections shown in FIGS. 1A-C.

The carrier element 31 may be of a generally uniform composition such that the outer surface 33 facilitates the retention of the information element 61 thereon. For example, the carrier element 31 may be of a composition that is not relatively dense but rather one providing a generally lightweight, porous structure so that the outer surface is similarly relatively porous, and therefore capable of better retaining certain types of materials 51 used to form the information element 61 thereon.

Returning to FIG. 1, the outer surface 33, or one or more of the major surfaces 34, 35 or edge surfaces 36, 37, 38, 39 thereof may be of a texture or treated or processed to facilitate the receipt and retention of the material 51 from which the information element 61 is formed. For example, the outer surface 33 may include adhesive properties as a result of applying heat, steam, pressure, and adhesive adjuncts to the carrier element 31. The adjuncts added to the surface 34 or incorporated into the carrier element 31 may be inherently sticky or tacky, or caused to sticky or tacky and may include food gums, hydrocolloids, oils, starches, proteins, cellulosics, sugars, fats, etc.

FIG. 2 illustrates the positioning of the material 51 from which the information element 61 is formed on major surface 34. While the discussion in the following will be in terms of providing an information element 61 on one surface 34 of the carrier element 31, the carrier element 31 may be of a composition and treated, and the information element 61 may be of a composition and treated such that an information element may be placed on one or more of the surfaces 34-39 (see FIG. 1). It will also be understood that the carrier element 31 may be one or more carrier element and the information element 61 may be one or more information element as desired, some examples of which are described herein.

In one example, FIG. 3 illustrates a carrier element 31 whose major surface 34 is treated such that positive surface elements 41 are formed thereon, and thereby a positive surface 43. For simplicity purposes, FIG. 3 shows how an image of two concentric circles that may be formed on the outer major surface 34 through the use of positive surface elements 41. However, information elements 61 having more complex imagery than that shown in FIG. 3 may be formed on the major outer surface 34 through the use of positive surface elements 41. The same or other imagery may be formed on any of the other major or edge surfaces 35-39 (see FIG. 1) through the use of positive surface elements 41. Positive surface elements 41 are those that facilitate the placement and retention of the information material 51 in and on the outer surface 33 of the carrier element 31 to form the positive surface 43.

In order to form positive surface elements 41, for example, on the major outer surface 34, the major outer surface 34 may be treated according to a variety of methods. Treatment, for example, may include applying surface material 42 to the surface 33. Such surface material from which the positive surface elements 41 may be formed may include that having a generally adhesive or sticky quality. Information material 51 can be of a composition and state such that when it is placed on surface material 42 the information material 51 generally adheres to or combines with the surface material 42. For such treatment, information material 51 may be in the form of a powder or spray and directed to make contact with the surface material 42 from which the positive surface elements 41 are formed. A material 51 having such a powdery or sprayable consistency would tend to adhere to the surface material 42 forming the positive surface element 41. With the elimination of any material 51 that did not adhere in such fashion, an information element 61 having the desired image will be formed on the outer surface 33 and not elsewhere.

Treatment of the outer surface 33 (or other of the surfaces 35-39) to define the positive surface elements 41, and thereby the positive surface area 43 for retention of the information material 51 thereon, can include also mechanical treatment of the outer surface 33 such as by abrading those areas that are to be the positive surface areas 41. Abrasion makes the areas 43 more porous than the remaining portions of the outer surface 33, and thereby facilitates the retention of certain types of information material 51 thereon. Fine-rained material 51 that is later processed to form a generally durable information element 61 may be used with such treated carrier elements 31.

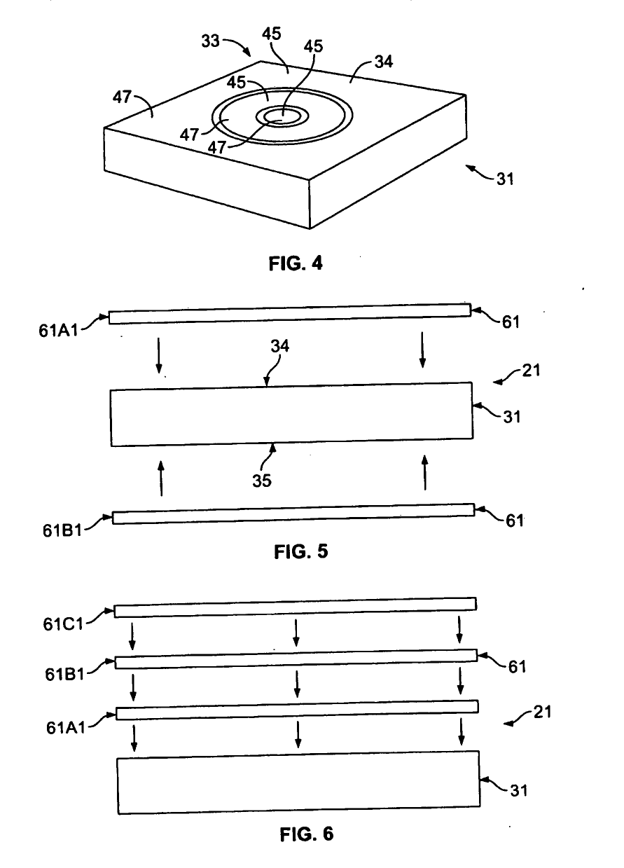

Alternatively, the carrier element major may include an outer surface 33 that is treated such that negative surface elements 45 are formed thereon and thereby a negative surface area 47. Again, and for simplicity purposes, FIG. 4 shows how an image of a hollow circle may be formed on the major surface 34 through the use of negative surface elements 45. However, information elements 51 having more complex imagery than that shown in FIG. 4 may be formed on the outer surface 33 through the use of negative surface elements 51. Negative surface elements 51 are those that facilitate the placement and retention of the Information material 51 in and on areas of the outer surface 33 outside the area 47 defined by the negative surface elements 45. In order to form negative surface elements 45, for example, on the major outer surface 34, the major outer surface 34 may be treated according to known methods. For example, a material—such as one that generally does not mix with a water based material—may be applied to the area 47 defined as the negative surface elements 45, thereby permitting only the area of the major outer surface 34 not so defined to accept the information material 51. One material by which the negative surface elements 45 and the negative surface area 47 may be formed includes an oil or oil-based material having sufficient viscosity such that it remains generally in the area 47.

The outer surface 33 may also be treated to define the positive surface elements 41 and thereby the positive surface area for retention of the information material 61. Such treatment includes mechanical treatment such as compressing the carrier element by the application of pressure on the negative pressure area 47, thereby making it more generally less porous than the remaining portions of the outer surface 33, and thereby less receptive to information material 51 that when appropriately composed and applied, will adhere to more porous areas. Compressing the carrier element 31 to give it an “embossed look” advantageously permits the formation of “3D” like effects on the surface. For example, this process may include stamping and pressing steps. Multiple information elements 61A1, 61B1, etc. (see FIGS. 10A-C) may be joined together through a variety of mechanisms and means.

Flavorings that may be included within an information element 61 or carrier element 31 can include natural or artificial flavorings in liquid form and/or solid form and other flavorings as onion powder, soy powder, tomato powder, or various agents that provide fragrance or perfume. Flavorings or additives when applied in dehydrated form to a carrier element 31 may be used in conjunction with some moisture to promote adherence to the carrier element 31. The carrier element 31 and information element may be processed through a variety of methods including frying, smoking, or texturing.

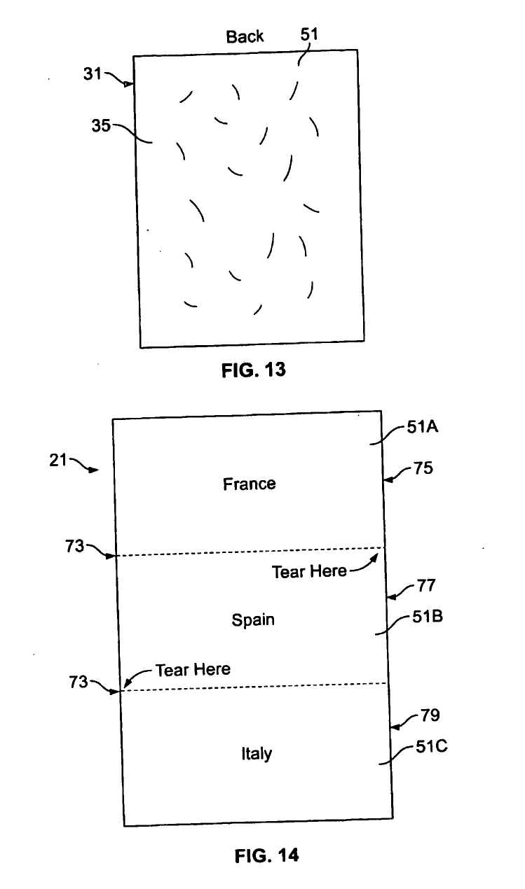

FIGS. 12-14 show additional embodiments of the present invention. The illustrated embodiments include information material 51 positioned on a carrier element such that an information element 61 is formed that at least in the form of writing. The information material 51 may be in the form of edible inks. Such embodiments can act as edible menus (FIGS. 12 and 13) or promotional materials discussing foods of different countries (FIG. 14).

FIG. 12 shows an embodiment in which the information material 51 is printed on a front surface 34 of the carrier element 31. An additional embodiment shown in FIG. 13 is formed from a carrier element 31 having one or more information materials 51—such as flavorings—layered onto a bottom surface 35.

FIG. 14 shows an embodiment in which a substitute food item 21 is subdivided into parts by one or more tine of weakened material 73. In this embodiment, each part 75, 77, 79 may include different information materials 51A, 51B, and 51C wherein each of the different information materials is unique from each other. In the illustrated embodiment, the information material 51A may correspond to a food found in France, the information material 51B may correspond to a food found in Spain, and the information material 51C may correspond to a food found in Italy, for example.

The substitute food items, including the carrier element and/or information element, may include one or more of the following ingredients: turmeric, curcumin, riboflavin (vitamin B2), tartrazine, alkanet, chrysoine resorcinol, Quinoline yellow, Yellow 2G, Sunset Yellow FCF, Orange GGN, Cochineal, carmines, Citrus red 2, Orcein, Orchil, azorubine, carmoisine, amaranth, Brilliant Scarlet 4R, Ponceau 4R, Ponceau SX, Scarlet GN, Ponceau 6R, erythrosine, Red 2G, Allura red AC, Indanthrene blue RS, Patent blue V, indigo carmine, indigotine, Brilliant blue FCF, chlorophylls and chlorophyllins, copper complexes of chlorophylls, Green S, Fast green FCF, caramel I—plain, caramel II—caustic sulfite process, caramel III—ammonia process, caramel IV—ammonia sulfite process, Black PN, Brilliant Black BN, Black 7984, Vegetable carbon, carbon black, Brown FK, Chocolate Brown HT, carotenes (alpha-carotene, beta-carotene, gamma-carotene), annatto, bixin, norbixin, paprika extract, capsanthin, capsorubin, lycopene, beta-apo-8′-carotenal (C 30), beta-apo-8′-carotenic acid ethyl ester, flavoxanthin, lutein, cryptoxanthin, rubixanthin, violaxanthin, rhodoxanthin, canthaxanthin, zeaxanthin, citranaxanthin, astaxanthin, beet red, beetroot red, betanin, anthocyanins, Saffron, crocetin and crocin, gardenia blue, sandalwood, calcium carbonates, chalk, titanium dioxide, iron oxides and hydroxides, aluminium, silver, gold, litholrubine, Lithol Rubine BK, Pigment Rubine, latolrubine, tannins, orchil, sorbic acid, sodium sorbate, potassium sorbate, calcium sorbate, heptyl p-hydroxybenzoate, benzoic acid, sodium benzoate, potassium benzoate, calcium benzoate, ethyl para-hydroxybenzoate, sodium ethyl para-hydroxybenzoate, propylparaben (propyl para-hydroxybenzoate), sodium propyl para-hydroxybenzoate, methylparaben (methyl para-hydroxybenzoate), sodium methyl para-hydroxybenzoate, sulfur dioxide, sodium sulfite, sodium bisulfite (sodium hydrogen sulfite), sodium metabisulfite, potassium metabisulfite, potassium sulfite, sodium sulfite, calcium sulfite, calcium bisulfite, calcium hydrogen sulfite, potassium bisulfite, potassium hydrogen sulfite, biphenyl, diphenyl, orthophenyl phenol, 2-hydroxybiphenyl, sodium orthophenyl phenol, thiabendazole, nisin, pimaricin, natamycin, formic acid, sodium formate, calcium formate, hexamine (hexamethylene tetramine), formaldehyde, gum guaicum, dimethyl dicarbonate, potassium nitrite, sodium nitrite, sodium nitrate, potassium nitrate, glacial acetic acid, potassium acetates, sodium acetate, sodium hydrogen acetate, calcium acetate, ammonium acetate, dehydroacetic acid, sodium dehydroacetate, lactic acid, propionic acid, sodium propionate, calcium propionate, potassium propionate, boric acid, sodium tetraborate, borax, carbon dioxide, malic acid, fumaric acid, ascorbic acid, sodium ascorbate, calcium ascorbate, potassium ascorbate, ascorbyl palmitate, ascorbyl stearate, tocopherol concentrate (natural), dl-alpha-tocopherol (synthetic), gamma-tocopherol(synthetic), delta-tocopherol(synthetic), propyl gallate, octyl gallate, dodecyl gallate, erythorbic acid, sodium erythorbate, erythorbin acid, sodium erythorbin, tert-butylhydroquinone, butylated hydroxyanisole (BHA), butylated hydroxytoluene (BHT), lecithins, sodium lactate, potassium lactate, calcium lactate, ammonium lactate, magnesium lactate, citric acid, sodium citrates, potassium citrates, calcium citrates, tartaric acid, sodium tartrates, potassium tartrates, potassium sodium tartrate, phosphoric acid, sodium phosphates, potassium phosphates, calcium phosphates, ammonium phosphates, magnesium phosphates, lecithin citrate, magnesium citrate, ammonium malate, sodium malates, potassium malate, calcium malates, metatartaric acid, calcium tartrate, adipic acid, sodium adipate, potassium adipate, ammonium adipates, succinic acid, sodium succinates, sodium fumarate, potassium fumarate, calcium fumarate, ammonium fumarate, 1,4-heptonolactone, niacin (nicotinic acid), nicotinamide (vitamin B3), triammonium citrate, ferric ammonium citrate, ammonium ferrocitrate, isopropyl citrates, calcium disodium EDTA, disodium ethylenediaminetetraacetate, oxystearin, thiodipropionic acid, dilauryl thiodipropionate, distearyl thiodipropionate, phytic acid, calcium lactobionate, alginic acid, sodium alginate, potassium alginate, ammonium alginate, calcium alginate, propylene glycol alginate, propane-1,2-diol alginate, agar, carrageenan, processed eucheuma seaweed, arabinogalactan, locust bean gum, guar gum, tragacanth, gum acacia, gum arabic, xanthan gum, karaya gum, tara gum, gellan gum, sorbitol, mannitol, glycerin, konjac, konjac gum, konjac glucomannate, polyoxyethylene (8) stearate, polyoxyethylene (40) stearate, polysorbate 20, polysorbate 80, polysorbate 40, polysorbate 60, polysorbate 65, pectin, gelatine, ammonium phosphatides, brominated vegetable oil, sucrose acetate isobutyrate, glycerol esters of wood rosin, diphosphates, triphosphates, polyphosphates, beta-cyclodextrin, powdered cellulose, microcrystalline cellulose, methylcellulose, hydroxypropyl cellulose, hydroxypropyl methylcellulose, methyl ethyl cellulose, ethyl methyl cellulose, sodium carboxymethylcellulose, crosslinked sodium carboxymethylcellulose, enzymatically hydrolyzed carboxymethyl cellulose, magnesium stearate, sodium, potassium and calcium salts of fatty acids, magnesium salts of fatty acids, mono- and diglycerides of fatty acids—glyceryl monostearate, glyceryl distearate, acetic acid esters of mono- and diglycerides of fatty acids, lactic acid esters of mono- and diglycerides of fatty acids, citric acid esters of mono- and diglycerides of fatty acids, tartaric acid esters of mono- and diglycerides of fatty acids, diacetyltartaric acid esters of mono- and diglycerides of fatty acids, mixed acetic and tartaric acid esters of mono- and diglycerides of fatty acids, sucrose esters of fatty acids, sucroglycerides, polyglycerol esters of fatty acids, polyglycerol polyricinoleate, propylene glycol esters of fatty acids, lactylated fatty acid esters of glycerol and propylene glycol, thermally oxidised soya bean oil, dioctyl sodium sulfosuccinate, sodium stearoyl lactylate, calcium stearoyl lactylate, stearyl tartarate, sorbitan monostearate, sorbitan tristearate, sorbitan monolaurate, sorbitan monooleate, sorbitan monopalmitate, sodium carbonate, sodium bicarbonate, potassium carbonate, potassium bicarbonate, ammonium carbonate, ammonium bicarbonate, magnesium carbonate, hydrochloric acid, potassium chloride, calcium chloride, ammonium chloride, magnesium chloride, stannous chloride, sulfuric acid, sodium sulfate, potassium sulfate, calcium sulfate, ammonium sulfate, magnesium sulfate, Epsom salts, cupric sulfate, aluminium sulfate, aluminium sodium sulfate, aluminium potassium sulfate, aluminium ammonium sulfate, sodium hydroxide, potassium hydroxide, calcium hydroxide, ammonium hydroxide, magnesium hydroxide, calcium oxide, magnesium oxide, sodium ferrocyanide, potassium ferrocyanide, calcium ferrocyanide, dicalcium diphosphate, sodium aluminium phosphate, bone phosphate, calcium polyphosphates, ammonium polyphosphates, silicon dioxide, calcium silicate, magnesium silicate, talc, sodium aluminosilicate (sodium aluminium silicate), potassium aluminium silicate, calcium aluminosilicate (calcium aluminium silicate), bentonite, kaolin, aluminium silicate, stearic acid, glucono-delta-lactone, sodium gluconate, potassium gluconate, calcium gluconate, ferrous gluconate, ferrous lactate, glutamic acid, monosodium glutamate (MSG), monopotassium glutamate, calcium diglutamate, monoammonium glutamate, magnesium diglutamate, guanylic acid, disodium guanylate, dipotassium guanylate, calcium guanylate, inosinic acid, disodium inosinate, dipotassium inosinate, calcium inosinate, calcium 5′-ribonucleotides, disodium 5′-ribonucleotides, maltol, ethyl maltol, glycine, leucine, zinc acetate, dimethylpolysiloxane, beeswax, candelilla wax, carnauba wax, shellac, paraffins, refined microcrystalline wax, montanic acid esters, oxidised polyethylene wax, L-cysteine, potassium bromate, chlorine, chlorine dioxide, carbamide, benzoyl peroxide, argon, helium, nitrogen, nitrous oxide, butane, isobutane, Acesulfame potassium, aspartame, cyclamic acid, cyclamates, isomalt, saccharin, sucralose, alitame, thaumatin, neohesperidin dihydrochalcone, maltitol, lactitol, xylitol, quillaia extract, choline salts and esters, amylases, glucose oxidase, invertase, lipases, lysozyme, polydextrose, poly vinyl pyrrolidone, polyvinylpolypyrrolidone, dextrin roasted starch, acid treated starch, alkaline treated starch, bleached starch, oxidised starch, enzyme treated starch, monostarch phosphate, distarch phosphate, phosphated distarch phosphate, acetylated distarch phosphate, acetylated starch, acetylated distarch adipate, hydroxypropyl starch, hydroxypropyl distarch phosphate, starch sodium octenylsuccinate, acetylated oxidised starch, triethyl citrate, ethanol, triacetin, propylene glycol, and polyethylene glycol 8000.

While the present inventions and what is considered presently to be the best modes thereof have been described in a manner that establishes possession thereof by the inventors and that enables those of ordinary skill in the art to make and use the inventions, it will be understood and appreciated that there are many equivalents to the exemplary embodiments disclosed herein and that myriad modifications and variations may be made thereto without departing from the scope and spirit of the inventions, which are to be limited not by the exemplary embodiments but by the appended claims.

What is claimed is:

1. A food emulator for the production of a substitute food item, comprising:
a control panel through which instructions are conveyed for operation of a discharge unit, said discharge unit including information material and positioned so that upon receipt of the instructions the information material is applied to a surface of a carrier element to form an information element thereon and thereby the substitute food item.

2. A method of forming an edible article for use as a substitute for a food item, comprising:
forming a carrier element of an edible substance; and
applying an information element onto a surface of the carrier element, wherein the information element includes organoleptic information related to the food item.

3. The food emulator according to claim 1 further comprising an intake component for receiving a food item for which the food emulator will produce the substitute food item.

4. The food emulator according to claim 1 further comprising a memory component to store said instructions.

5. The food emulator according to claim 3 further comprising a processor by which one or more characteristics of the food item is processed.

6. The food emulator according to claim 1 further comprising a recorder to accept consideration for the vending of the substitute food item.

7. The food emulator according to claim 5, wherein said processor further includes an identifier by which the presence and identity of the food item may be identified to said control panel.

8. The method of claim 2 further comprising the step of placing an edible infrared material on the carrier element.

9. The method of claim 2 further comprising the step of shaping the carrier element.

10. The method of claim 2 further comprising the step of positioning a gel layer between the carrier element and the information element.

11. The method of claim 2, wherein said step of applying an information element further comprises spraying the information element onto a surface of the carrier element.

12. The method of claim 2, wherein said step of applying an information element further comprises printing the information element onto a surface of the carrier element.

13. An edible article that can substitute for a food item, comprising:
a carrier element wherein said carrier element is an edible material;
an information element disposed on one or more surfaces of said carrier element wherein said information element conveys information about the food item.

14. The edible article of claim 13 further comprising an edible infrared material positioned on said carrier element.

15. The edible article of claim 13 further comprising an edible infrared material positioned on said information element.

16. The edible article of claim 13 further comprising an attaching means such that the edible article can be easily secured or removed from other surfaces.

17. The edible article of claim 13 further comprising a use component wherein said use component provides support for or permits the shaping of the edible article.

18. The edible article of claim 17, wherein said use component is a structural element.

19. The edible article of claim 17, wherein said use component is formed from generally adhesive edible substances.

20. The edible article of claim 13 further comprising a gel layer positioned between said carrier element and said information element.

21. The edible article of claim 13 further comprising a positive surface element to facilitate the placement and retention of said information element.

22. The edible article of claim 13 further comprising a negative surface element to facilitate the placement and retention of said information element.

23. An apparatus for producing a substitute food item from a food item, comprising:
an intake component for receiving the food item;
a processor by which one or more characteristics of the food item is processed; and
a communication element to convey instructions to the apparatus to form the substitute food item.

24. The apparatus of claim 23 further comprising a memory component to store said instructions.

25. The apparatus of claim 23 further comprising a storage unit including information material for placement onto a carrier element.

26. The apparatus of claim 23, wherein said communication element is a control panel.

27. The apparatus of claim 23, wherein said communication element includes a receiver and a transmitter.

28. The apparatus of claim 23, wherein said processor further includes an identifier by which the presence and identity of the food item may be identified to said communication element.

29. The apparatus of claim 23 further comprising a recorder to accept consideration for the vending of the substitute food item.

30. A method of producing a substitute food item from a food item, comprising:
placing the food item into an intake component;
processing one or more characteristics of the food item; and
conveying instructions to form the substitute food item.

31. The method of claim 30, wherein said step of conveying instructions to form the substitute food item further comprises the steps of:
forming a carrier element of an edible substance; and
applying an information element onto the surface of the carrier element.
